(19) United States
(12) Patent Application Publication
(10) Pub. No.: US 2021/0138119 A1
(43) Pub. Date: May 13, 2021

(54) METHOD FOR USING A BIODEGRADABLE METAL ALLOY TO ANCHOR DETACHED TISSUE TO HARD TISSUE

(71) Applicant: NATIONAL CHENG KUNG UNIVERSITY, TAINAN CITY (TW)

(72) Inventors: MING-LONG YEH, TAINAN CITY (TW); CHIEN-YU LIN, TAINAN CITY (TW); WEI-REN SU, TAINAN CITY (TW); FEI-YI HUNG, TAINAN CITY (TW)

(21) Appl. No.: 17/101,196

(22) Filed: Nov. 23, 2020

Related U.S. Application Data
(62) Division of application No. 16/211,737, filed on Dec. 6, 2018.

(51) Int. Cl. A61L 31/02; A61L 31/14; C22C 23/00

(52) U.S. Cl. CPC A61L 31/022; A61F 2/0811; C22C 23/00; A61L 31/148

(57) ABSTRACT

A biodegradable metal alloy for anchoring detached tissue to hard tissue, a method for making the same and a process for using the same are revealed. The biodegradable metal alloy includes a magnesium-zinc-zirconium (Mg—Zn—Zr) alloy and a magnesium fluoride (MgF₂) coating over the Mg—Zn—Zr alloy. The Mg—Zn—Zr alloy is a magnesium alloy containing 2.5-6.0 wt % zinc (Zn) and 0.42-0.80 wt % zirconium (Zr). Thereby the present biodegradable metal alloy not only provides sufficient fixation strength for anchoring detached tissue to hard tissue effectively but also promotes the bone growth and avoids metal/alloy artifacts in images.

### REFERENCE TO RELATED APPLICATIONS

This Application is being filed as a Divisional Application of patent application Ser. No. 16/211,737, filed 6 Dec. 2018, currently pending.

### BACKGROUND OF THE INVENTION

### Field of the Invention

The present invention relates to a biodegradable metal alloy for anchoring detached tissue to hard tissue, a method for making the same and a process for using the same, especially to a biodegradable metal alloy for effectively anchoring detached tissue to hard tissue, a method for making the same and a process for using the same that not only promote bone growth but also avoid metal/alloy artifacts in images.

### Description of Related Art

Suture anchor technique is a way that re-attaches detached tissues to hard tissues by inserting anchors with sutures into bone tissues, and the sutures are passed through the detached tissues to move the detached tissues back to its insertion site, and then secure the detached tissues onto the position. Thus, the contact area between the tissues is increased for promoting tissue regeneration and healing.

The most commonly used materials for suture anchors are titanium alloys at first. Refer to Chinese Pat. App. Pub. No. CN203074848U, an implantable bone anchor is revealed. The bone anchor features on an implantable anchor body provided with a threaded hole on an upper part thereof and spring threads on a lower part thereof. A bone graft hole is arranged at the implantable anchor body. The implantable anchor body is threaded into the bone at the injured area by the spring threads and a cutting edge on the front end of the spring threads for fixing. Then bone fragments are filled into the bone graft hole and inserted by sutures to connect with soft tissues tightly. Thereby soft tissues of joints and bones are reconnected. Although titanium alloys provide good fixation, these non-biodegradable materials will be left in the body permanently once there is no second surgery for implant removal. The anchors may cause some adverse effects such as chronic local inflammatory reactions and the loosening or migration of anchors may cause cartilage damage. Moreover, the assessment of medical images is affected by titanium alloys. For example, the accuracy of magnetic resonance imaging (MRI) would be affected by metals, which is so called metallic artifacts. Thus, titanium alloys have been gradually replaced by polymers since metals interfere with imaging. However, as permanent implants, the mechanical properties of bioinert polymers are not as good as metal/alloy and they may cause consistent foreign body reactions in human bodies. According to many case reports, the use of bioinert polymers tends to cause several complications such as osteolysis, cyst formation, etc. As to degradable polymers, they can be absorbed over time in human bodies, but the degradable polymers have certain shortcomings. The degradable polymers have poor mechanical performance compared with metal/alloy. The degradation products of degradable polymers will ultimately lead to acidic micro-environment, which is harmful to tissue regeneration and tends to induce inflammatory reactions around the implants.

Thus, there is room for improvement and there is a need to provide a novel material for anchors used for securing detached tissue to hard tissue.

### SUMMARY OF THE INVENTION

Therefore, it is a primary object of the present invention to provide a biodegradable metal alloy for anchoring detached tissue to hard tissue, a method for making the same and a process for using the same. The biodegradable metal alloy can not only secure detached soft tissue such as anterior cruciate ligament to hard tissue effectively and provide sufficient fixation strength but also avoid metal/alloy artifacts in images. The degradation products of the biodegradable metal alloy can further enhance tissue healing.

In order to achieve the above object, a biodegradable metal alloy for anchoring detached tissue to hard tissue according to the present invention includes a magnesium-zinc-zirconium (Mg—Zn—Zr) alloy and a magnesium fluoride (MgF₂) coating over the Mg—Zn—Zr alloy. The Mg—Zn—Zr alloy is a magnesium alloy containing 2.5-6.0 wt % (weight percent) zinc (Zn) and 0.42-0.80 wt % zirconium (Zr). The Mg—Zn—Zr alloy is selected from the group consisting of ZK50, ZK30, ZK60, ZK51A-T5, ZK61-T5, ZK61-T6, ZK31-T5, ZK60-T5, ZK61-T5, ZK21A-F, ZK31-T5, ZK40A-T5, ZK60A-T5, ZK61, ZK50, ZK60-F, ZK60-T4, and ZK60-T6.

In order to achieve the above object, a method for making a biodegradable metal alloy for anchoring detached tissue to hard tissue according to the present invention includes the steps of (a) selecting a magnesium-zinc-zirconium (Mg—Zn—Zr) alloy that is a magnesium alloy containing 2.5-6.0 wt % zinc (Zn) and 0.42-0.80 wt % zirconium (Zr); and (b) immersing the Mg—Zn—Zr alloy in a 42% hydrogen fluoride (HF) solution and shaking the solution with the alloy therein for 24 hours to form a magnesium fluoride (MgF₂) coating over the Mg—Zn—Zr alloy.

In order to achieve the above object, a process for using a biodegradable metal alloy to anchor detached tissue to hard tissue according to the present invention includes inserting a biodegradable metal alloy into hard tissue with the free ends of sutures extending out of the hard tissue, and repairing the detached tissue to hard tissue by using Mason-Allen stitch. The biodegradable metal alloy is used for producing biodegradable anchors that secure detached tissue to hard tissue.

Thereby the biodegradable metal alloy of the present invention not only has high mechanical strength and ability to be absorbed by human bodies but also avoids metallic artifacts on interpretation of medical images (such as MRI) caused by titanium alloy, foreign-body reactions and tissue inflammation. In practice, the biodegradable metal alloy can be applied to arthroscopic surgery for repairing detached tissues such as rotator cuff tear in the shoulder.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a sectional view of an embodiment of biodegradable metal alloy used for anchoring detached tissue on hard tissue according to the present invention;

FIG. 2 is a schematic drawing showing an embodiment of biodegradable metal alloy used for anchoring detached tissue on hard tissue according to the present invention;

FIG. 3 shows results of corrosion resistance analysis of ZK50 alloy and MgF₂-ZK50 alloy of an embodiment according to the present invention;

FIG. 4 shows results of hydrogen release tests of ZK50 alloy and MgF₂-ZK50 alloy of an embodiment according to the present invention;

FIG. 5 shows results of cell cytotoxicity tests of ZK50 alloy and MgF₂-ZK50 alloy of an embodiment according to the present invention;

FIG. 6 shows scanning electron micrographs of ZK50 alloy and MgF₂-ZK50 alloy on which cells have been seeded for 4 hours, magnified 1000× of an embodiment according to the present invention;

FIG. 7 shows scanning electron micrographs of ZK50 alloy and MgF₂-ZK50 alloy on which cells have been seeded for 4 hours, magnified 2000× of an embodiment according to the present invention;

FIG. 8 shows scanning electron micrographs of ZK50 alloy and MgF₂-ZK50 alloy on which cells have been seeded for 24 hours, magnified 1000× of an embodiment according to the present invention;

FIG. 9 shows scanning electron micrographs of ZK50 alloy and MgF₂-ZK50 alloy on which cells have been seeded for 24 hours, magnified 2000× of an embodiment according to the present invention;

FIG. 10 is a schematic drawing showing an embodiment being applied to a biodegradable anchor according to the present invention;

FIG. 11 is a schematic drawing showing a pullout test of an embodiment according to the present invention;

FIG. 12 shows results of pullout tests of an embodiment of biodegradable metal alloy anchors and titanium alloy anchors according to the present invention;

FIG. 13 is a schematic drawing showing an embodiment tested on supraspinatus tendon of rotator cuff on shoulder joint of New Zealand White rabbit according to the present invention;

FIG. 14 shows computed tomography (CT) results of an embodiment of biodegradable metal alloy anchors, titanium alloy anchors, and surrounding tissues one month after surgery according to the resent invention;

FIG. 15 shows computed tomography (CT) results of an embodiment of biodegradable metal alloy anchors, titanium alloy anchors, and surrounding tissues three months after surgery according to the resent invention;

FIG. 16 shows tissue slices of tendon and bone implanted with an embodiment of a biodegradable metal alloy anchor and a titanium alloy anchor three months after surgery according to the present invention.

### DETAILED DESCRIPTION OF THE PREFERRED EMBODIMENT

Refer to FIG. 1, a biodegradable metal alloy 1 for anchoring detached tissue to hard tissue according to the present invention includes a magnesium-zinc-zirconium (Mg—Zn—Zr) alloy 11 and a magnesium fluoride (MgF₂) coating 12 over the Mg—Zn—Zr alloy 11. The Mg—Zn—Zr alloy 11 can be a magnesium alloy containing 2.5-6.0 wt % zinc (Zn) and 0.42-0.80 wt % zirconium (Zr).

Refer to FIG. 2, in a preferred embodiment, the present biodegradable metal alloy 1 is applied to a biodegradable anchor used for anchoring detached tissue to hard tissue and the biodegradable anchor is coated with a layer of MgF₂.

A method for making a biodegradable metal alloy for anchoring detached tissue to hard tissue according to the present invention includes the steps of (a) selecting a magnesium-zinc-zirconium (Mg—Zn—Zr) alloy. The Mg—Zn—Zr alloy can be a magnesium alloy containing 2.5-6.0 wt % zinc (Zn) and 0.42-0.80 wt % zirconium (Zr); and (b) immersing the Mg—Zn—Zr alloy in a 42% hydrogen fluoride (HF) solution and shaking the solution with the alloy therein for 24 hours to form a magnesium fluoride (MgF₂) coating over the Mg—Zn—Zr alloy.

A process for using a biodegradable metal alloy to anchor detached tissue to hard tissue according to the present invention includes inserting a biodegradable metal alloy into hard tissue with the free ends of sutures extending out of the hard tissue, and repairing the detached tissue to hard tissue by using Mason-Allen stitch. The biodegradable metal alloy includes a magnesium-zinc-zirconium (Mg—Zn—Zr) alloy and a magnesium fluoride (MgF₂) coating over the Mg—Zn—Zr alloy. Preferably, the Mg—Zn—Zr alloy is a magnesium alloy containing 2.5-6.0 wt % zinc (Zn) and 0.42-0.80 wt % zirconium (Zr). For example, the Mg—Zn—Zr alloy is a ZK50 alloy containing 5.0 wt % zinc and 0.5 wt % zirconium.

It should be noted that all mechanical properties of the alloy are decreased when the amount of zinc contained in the magnesium alloy is over a certain level such as 6.0 wt %. Moreover, microporosity occurs in the alloy and hot cracking tendency increases owing to a high amount of zinc. Once the amount of zinc contained in the magnesium alloy is below a certain level such as 2.5 wt %, the reduced corrosion resistance of the alloy caused by metal impurities such as iron, nickel etc. is unable to be improved. Thus, the amount of the zinc in the magnesium alloy is preferably ranging from 2.5% to 6.0 wt %. Moreover, the magnesium-zinc alloy with a higher zirconium content is difficult to process and cast. For example, the zirconium content is above 0.80 wt %. Yet the amount of zirconium contained in magnesium-zinc alloy is usually higher than 0.42 wt % for grain refining of zirconium. Thus, the amount of zirconium contained in magnesium-zinc alloy is preferably ranging from 0.42% to 0.80 wt %.

In a preferred embodiment, the Mg—Zn—Zr alloy is selected from ZK50, ZK30, ZK60, ZK51A-T5, ZK61-T5, ZK61-T6, ZK31-T5, ZK60-T5, ZK61-T5, ZK21A-F, ZK31-T5, ZK40A-T5, ZK60A-T5, ZK61, ZK50, ZK60-F, ZK60-T4, and ZK60-T6.

Magnesium alloy has good biocompatibility, light weight similar to bone mass and good mechanical properties. Thus, it is used as a new generation of biodegradable medical implants in recent years. According to the previous tests, magnesium alloys containing other metals have different properties such as rigidity, degree of degradation. Thus not all magnesium alloys can be applied to anchors for securing detached tissue to hard tissue. For example, magnesium alloys containing aluminum have shown to cause neurotoxicity after being implanted into human bodies. In order to develop materials suitable for fixing soft tissues and hard tissues, performance tests for these materials were carried out and analyzed.

### Embodiment One: Material Performance Test

In this embodiment, the biodegradable metal alloy used is Mg—Zn—Zr alloy, ZK50 (Zn 5.0 wt %, Zr 0.5 wt %) and the sample has a diameter of 1.2 cm and a thickness of 0.4 cm. The ZK50 alloy was immersed in a 42% hydrogen fluoride (HF) solution and the solution with the alloy sample therein was shaken for 24 hours to form a magnesium fluoride (MgF₂) coating for increasing the corrosion resistance. Refer to FIG. 2, the final product-MgF₂-ZK50 with black coating (MgF₂) is shown.

### 1. Corrosion Resistance Analysis

Electrochemical tests were performed for corrosion resistance analysis. The corrosion current density was determined by the potentiodynamic polarization curve. More specifically, potentiodynamic polarization tests were conducted at the scan rate of 0.001 V/s and the scan range of −2 V to −1 V. The test temperature was controlled at room temperature (about 25° C.) and the solution used was revised simulated body fluid (r-SBF). The electrochemical tests were performed on the surface of the samples to estimate the corrosion property of them. After scanning, the data obtained was plotted to form FIG. 3 while related parameters such as corrosion potential (E_{corr}) and corrosion current density (I_{corr}) obtained are shown in Table 1. Refer to Table 1, the corrosion current density of MgF₂-ZK50 alloy (with MgF₂ coating) is 5 times lower than that of ZK50 alloy. Thus the MgF₂-ZK50 alloy obtained after surface modification has better anticorrosion performance.

TABLE 1

| | E_{corr} (V) | I_{corr} (μA/cm²) |
|---|---|---|
| ZK50 alloy | −1.563 | 65.492 |
| MgF₂-ZK50 alloy | −1.451 | 12.878 |

### 2. Hydrogen Release Test

Hydrogen gas is released during corrosion of magnesium alloy. Thus the larger volume the hydrogen gas released represents the lower corrosion resistance of the alloy. The test results are shown in FIG. 4, there is a significant difference between the sample of ZK50 alloy without surface modification and the sample of MgF₂-ZK50 alloy with surface modification. The hydrogen releasement of the sample of MgF₂-ZK50 alloy reached saturation after 12-hour immersion, but the hydrogen production of the sample of ZK50 alloy increased constantly owing to consistent corrosion.

### 3. Cytotoxicity Test

Testing for cytotoxicity is an important step toward ensuring the material's biocompatibility. ZK50 alloy and MgF₂-ZK50 alloy were immersed into cell culture medium respectively and placed in an incubator (5% CO₂ & 37° C.) for 24 hours. Then extraction was carried out to get extracted medium of ZK50 alloy and extracted medium of MgF₂-ZK50 alloy. Human osteoblast-like MG63 cells were cultured in the extracted medium of ZK50 alloy, the extracted medium of MgF₂-ZK50 alloy, and culture medium only (as the control group), respectively. The cell viability in all groups from day 1 to day 7 was tested and the results are shown in FIG. 5. Both the ZK50 group and the MgF₂-ZK50 group showed no cytotoxicity (data represented as the mean±SEM (n=5) and * means p<0.05). During the culture, the highest cell viability was shown in the MgF₂-ZK50 group while the difference between the MgF₂-ZK50 group and other groups was the most obvious at day 1. The cell viability of the ZK50 group was declined at day 7 but no cytotoxicity was shown compared with the control group. The cell viability in the MgF₂-ZK50 group was higher than that in the ZK50 group and the control group so that the MgF₂ coating could stimulate cell growth.

### 4. Cell Adhesion Test

In order to learn adhesion of the osteoblasts on surface of various materials or implants, the cell adhesion morphology was analyzed through observation of scanning electron microscope (SEM). Human osteoblast-like MG63 cells derived from an osteosarcoma were seeded on both the ZK50 surface and the MgF₂-ZK50 surface for 4 hours and 24 hours, respectively and then fixed and dehydrated in turn. The test results are shown in FIG. 6 and FIG. 7, cells seeded on the ZK50 alloy without any coating became more spherical after 4 hours. Moreover, obvious defects such as cracks were observed on the surface of the ZK50 alloy. This means ZK50 alloy is easily corroded by the cell culture medium. As to the MgF₂-ZK50 with coating, the cell morphology therein was better owing to the flattened shape and extension of pseudopodia and the alloy surface was protected by the MgF₂ coating. Thus the SEM results showed that MgF₂-ZK50 modified by hydrogen fluoride can stimulate cells to have good initial cell adhesion.

Refer to FIG. 8 and FIG. 9, after the cells were in contact with the alloy surface for 24 hours, the number of cells on surface of the ZK50 alloy was fewer than that on surface of the MgF₂-ZK50 alloy and the cracks appeared due to corrosion of the cell culture medium became bigger. Moreover, the cell morphology in the ZK50 group showed ordinary adhesion structure of MG63 osteoblast-like cells that is spindle-shaped. The cell morphology in the MgF₂-ZK50 group seen as spindle and flat means that some cells might be already adhered to the surface completely. The cells in this group also extended filopodia to reach cells at the distal end. Thus the MgF₂ coating provides a favorable environment for cell adhesion and growth.

### Embodiment Two

In this embodiment, the biodegradable metal alloy used is Mg—Zn—Zr alloy, ZK50 alloy (Zn 5.0 wt %, Zr 0.5 wt %). The metal alloy was processed by the computer numerical control (CNC) machine to get magnesium alloy anchors with proper size for being applied to New Zealand White rabbits' shoulders. Then the ZK50 anchors were immersed in a 42% hydrogen fluoride (HF) solution and the solution with the alloy anchors therein was shaken for 24 hours to form a magnesium fluoride (MgF₂) coating on the anchor for increasing the corrosion resistance. The anchor produced is shown in FIG. 10 and the black coating (MgF₂) on the surface thereof can not only stimulate bone growth but also improve corrosion resistance and degradation of Mg—Zn—Zr alloy. Then two non-degradable sutures were passed through the eyelet of the anchor for securing detached tissue to hard tissue.

In animal tests, magnesium alloy anchors made of biodegradable metal alloy MgF₂-ZK50 and titanium anchors (Ti6Al4V) with sutures were implanted into animals first and then harvested with the hard tissue surrounding the anchor as a whole to perform pullout tests.

Pullout tests were conducted on euthanized rabbits' shoulders (n=5) to learn initial fixation of torn tendon provided by different anchors. Two anchors including Ti6Al4V anchor and MgF₂-coated ZK50 (MgF₂-ZK50) anchor were implanted into the left limb and the right limb, respectively of the rabbit and each anchor was provided with sutures (FiberWires). After the anchor being implanted into the humeral head of rabbit's shoulder, only the humerus with the suture anchor was harvested from the rabbit to perform pullout test for reducing the influence of the tendons with different properties. As shown in FIG. 11, a part of the humerus further away from the position where the suture anchor was implanted was embedded in cement inside a PVC tube cap firstly. Then a cylindrical jig was used to hold the midportion of the PVC tube cap with the humeral bone inside. The standard distance between the eyelet of the anchor and the jig was 50 mm. An axial force was applied to the FiberWires and the vertical displacement per minute was measured to get the pullout strength. The pullout direction and the axis of the humeral shaft were parallel. The constant pullout rate was set at 5 mm/min. The load-displacement curve was recorded by a computerized monitor at room temperature.

Refer to FIG. 12 and Table 2, the test results were presented as the mean±SD. Statistical analysis was performed using GraphPad Prism. The two groups were compared using a paired two-tailed Student's t-test. Compared with biodegradable polymer, the possibility of anchor breakage in the failure mode of the metal group is much lower owing to excellent mechanical properties. The results showed that the average value of the pullout strength of MgF₂-coated anchors is a bit higher than that of Ti6Al4V anchors, but there is no significant difference between them (p=0.2899).

TABLE 2

| | Sample | Pullout strength (N) |
|---|---|---|
| Titanium alloy | 1 | 92.235 (Anchor pullout) |
| (Ti6Al4V) | 2 | 154.77 (Suture breakage) |
| | 3 | 109.34 (Anchor pullout) |
| | 4 | 102.68 (Anchor pullout) |
| | 5 | 116.35 (Suture breakage) |
| biodegradable metal | 1 | 109.59 (Anchor pullout) |
| alloy (magnesium | 2 | 140.13 (Anchor pullout) |
| alloy) | 3 | 98.239 (Anchor pullout) |
| (MgF₂-ZK50) | 4 | 162.34 (Suture breakage) |
| | 5 | 150.06 (Anchor pullout) |

### Embodiment Three

Refer to FIG. 13, the supraspinatus tendon of rotator cuff on shoulder joint of New Zealand White rabbit was fully cut and then was repaired and fixed to the original position by two sutures and Mason-Allen suture technique.

During animal testing, the recovery of rotator cuff repair in two groups implanted with magnesium anchor (MgF₂-ZK50) made of biodegradable metal alloy and titanium anchor (Ti6Al4V) made of conventional titanium alloy, respectively after surgery was compared.

The tendon and bone tissues containing the anchor are harvested 1 month after surgery and 3 months after surgery, respectively, and the recovery of the tissues was observed. Refer to FIG. 14, computed tomography (CT) results showed that the biodegradable MgF₂-ZK50 anchor remained intact after being implanted for 1 month. The implant showed good corrosion resistance at the beginning of implantation, the surrounding bone tissues regenerated quite well and attached to the anchor tightly. In contrast with the MgF₂-ZK50 anchor, the bone mass around the Ti6Al4V anchor looked more porous. This is due to stress shielding caused by the modulus mismatch between the titanium alloy and the surrounding bone tissues.

Refer to FIG. 15, there is no significant difference in the titanium alloy (Ti6Al4V) group three months after implantation, compared with the image taken one month after implantation. As to the biodegradable MgF₂-ZK50 anchor, it was observed that the magnesium alloy was gradually degraded and absorbed by surrounding tissues. The space where the implant was placed in was filled with new bone.

As shown in FIG. 16, the tendon was tightly attached to the bone in both the titanium alloy (Ti6Al4V) group and the magnesium alloy (MgF₂-ZK50) group. Yet the comparison indicated good compatibility between degraded products of the magnesium alloy (MgF₂-ZK50) and bone mass. This has proven the feasibility of using magnesium alloy (MgF₂-ZK50) anchors on repairing detached tissues and hard tissues.

Compared with techniques available now, the present invention has the following advantages:
1. The present biodegradable metal alloy can provide good degradation efficiency and promote bone growth. After the metal alloy being absorbed by human bodies, the bone tissues grow into the space where the implant was placed in before.
2. The present biodegradable metal alloy provides sufficient fixation strength to make the detached soft tissues contact with hard tissues so as to accelerate tissue healing.
3. The present biodegradable metal alloy and cortical bone have similar mechanical properties. The metal alloy can be degraded over time in human bodies so as to avoid metal/alloy artifacts in images.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A process for using a biodegradable metal alloy to anchor detached tissue to hard tissue comprising: inserting a biodegradable metal alloy into a hard tissue with free ends of sutures extending out of the hard tissue, and repairing the detached tissue to the hard tissue by using a Mason-Allen stitch; wherein the biodegradable metal alloy includes a magnesium-zinc-zirconium (Mg—Zn—Zr) alloy and a magnesium fluoride (MgF₂) coating over the Mg—Zn—Zr alloy; the Mg—Zn—Zr alloy is a magnesium alloy containing 2.5-6.0 wt % zinc (Zn) and 0.42-0.80 wt % zirconium (Zr).

2. The process as claimed in claim 1, wherein the Mg—Zn—Zr alloy is selected from the group consisting of ZK50, ZK30, ZK60, ZK51A-T5, ZK61-T5, ZK61-T6, ZK31-T5, ZK60-T5, ZK61-T5, ZK21A-F, ZK31-T5, ZK40A-T5, ZK60A-T5, ZK61, ZK50, ZK60-F, ZK60-T4, and ZK60-T6.
